(19) United States
(12) Patent Application Publication
(10) Pub. No.: US 2025/0241671 A1
(43) Pub. Date: Jul. 31, 2025

(54) MEDICAL SYSTEMS AND DEVICES FOR TISSUE MARKING AND/OR REMOVAL AND RELATED METHODS

(71) Applicant: Boston Scientific Scimed, Inc., Maple Grove, MN (US)

(72) Inventors: Samuel GOSHEN, Brookline, MA (US); Siavash Sam PARKHIDEH, Setauket, NY (US); Francini ZUNIGA LUNA, Clinton, MA (US)

(73) Assignee: Boston Scientific Scimed, Inc., Maple Grove, MN (US)

(21) Appl. No.: 19/041,187

(22) Filed: Jan. 30, 2025

Related U.S. Application Data
(60) Provisional application No. 63/627,173, filed on Jan. 31, 2024.

(51) Int. Cl. A61B 17/32; A61B 90/00

(52) U.S. Cl. CPC A61B 17/320016; A61B 90/39; A61B 2017/320064; A61B 2090/395; A61B 2217/005

(57) ABSTRACT

Medical systems and devices for tissue marking and/or removal and related methods are described herein. The disclosure includes a medical device. The medical device includes a receiving portion. The receiving portion includes a vessel. The vessel includes an opening. The receiving portion includes a blade. The blade includes a first sharp edge on a first side and a second sharp edge on a second side opposite to the first side. The receiving portion includes a first configuration and a second configuration. When the receiving portion is in the first configuration, the first sharp edge extends from one side of the opening. When the receiving portion is in the second configuration, the second sharp edge extends from another side of the opening.

### CROSS-REFERENCE TO RELATED APPLICATIONS

This application claims the benefit of priority to U.S. Provisional Application No. 63/627,173, filed on Jan. 31, 2024, which is incorporated herein by reference in its entirety.

### TECHNICAL FIELD

Various aspects of this disclosure generally relate to medical systems and devices for tissue marking and/or removal and related methods. In particular, aspects of this disclosure relate to medical systems and devices for, for example, coloring, cauterizing, manipulating, resecting, grasping, and/or collecting tissue, such as, for example, marking and/or capture devices.

### BACKGROUND

Medical devices, such as endoscopes or other suitable introduction devices, are employed for a variety of diagnostic and surgical procedures, such as endoscopy, laparoscopy, arthroscopy, gynoscopy, thoracoscopy, cystoscopy, etc. Many of these procedures are carried out for purposes of tissue resection, which may include removal of tissue of an organ or a gland to treat tumors, infestations, and the like. In particular, such procedures may be carried out by inserting an insertion device into a patient's body through a surgical incision, or via a natural anatomical orifice (e.g., mouth, vagina, or rectum), and performing the procedure or operation.

Snare devices, hemostasis clips, and biopsy forceps have been used in many medical procedures, including Endoscopic Mucosal Resection (EMR) and Endoscopic Sub-mucosal Resection (ESR), polypectomy, mucosectomy, etc., for resecting tissue from a treatment site. Other devices that may be used include graspers, baskets, probes, scissors, retrieval devices, lasers, and other tools, into the subject's body. However, when removing tissue from a treatment site (e.g., a portion of the colon), there is a risk of incomplete removal of the tissue (e.g., a cancerous polyp). Incomplete removal of the tissue (i.e., incomplete removal of a polyp) may be a result of when a medical professional misjudges the boundary of problematic tissue and/or is unable to see the boundary while operating.

The medical systems, devices, and methods of this disclosure may help to rectify some of the deficiencies described above or address other aspects of the art.

### SUMMARY

Each of the aspects disclosed herein may include one or more of the features described in connection with any of the other disclosed aspects.

For example, the disclosure includes a medical device. The medical device may include a receiving portion. The receiving portion may include a vessel. The vessel may include a blade. The blade may include a first sharp edge on a first side and a second sharp edge on a second side opposite to the first side. The receiving portion may include a first configuration and a second configuration. When the receiving portion is in the first configuration, the first sharp edge may extend from one side of the opening. When the receiving portion is in the second configuration, the second sharp edge may extend from another side of the opening.

The medical device may include one or more of the following features. The blade may be pivotable about a proximalmost point of the opening and a distalmost point of the opening. The blade may be operably connected to an actuator. Actuating the actuator may transition the receiving portion from the first configuration to the second configuration. The blade may travel along an interior surface of the vessel while the receiving portion transitions from the first configuration to the second configuration.

The medical device may include one or more of the following features. When in the first configuration, the blade may be configured to cut a portion of a marker into a treatment site. When in the second configuration, the blade may be configured to cut another portion of the marker into the treatment site. The marker may include a circular shape or an elliptical shape.

The medical device may include one or more of the following features. The medical device may include an articulation portion including a pivot and an arm. A proximal end of the arm may be pivotally connected to the pivot. A distal end of the arm may be connected to a proximal end of the vessel. The pivot may be operably connected to a first actuator. Actuating the first actuator may pivot the arm along a horizontal plane. The pivot may be operably connected to a second actuator. Actuating the second actuator may pivot the arm along a vertical plane. The receiving portion may be operably connected to a third actuator. Actuating the third actuator may transition the receiving portion from the first configuration to the second configuration. Deactuating the third actuator may transition the receiving portion from the second configuration to the first configuration.

The medical device may include one or more of the following features. The vessel may include a semi-spherical shape or a semi-ellipsoidal shape. The opening may include a circular or elliptical shape. The blade may include an arcuate shape. A first longitudinal end of the blade may be pivotally connected to a proximalmost point of the opening and a second longitudinal end of the blade may be pivotally connected to a distalmost point of the opening.

The disclosure also includes a medical device. The medical device may include a container. The container may include a chamber and an opening at a distal end of the chamber. The opening may include a rim extending along a perimeter of the opening. The medical device may include a shaft. A distal end of the shaft may be connected to a proximal end of the container. The rim may be configured to apply a material or deliver energy to form a marker on a treatment site surrounding a target.

The medical device may include one or more of the following features. The medical device may be configured to be coupled to a suction source. The shaft may include an internal lumen that fluidly connects the suction source to the chamber of the container. The suction source may be configured to create negative pressure within the chamber of the container. The rim may be coated in a dye or marker pigment. The marker may be formed from the dye or marker pigment. The medical device may include an electrical conduit that is electrically connected to the rim. The electrical conduit may be configured to be coupled to an energy source. The rim may be configured to be energized by energy delivered by the electrical conduit.

This disclosure further includes a medical system. The medical system may include an insertion device. The insertion device may include a handle. The handle may include a port. The insertion device may include a shaft. The shaft may extend from a proximal end of the handle. The shaft may include a working channel. The medical system may include a medical device. The medical device may include an actuator positioned at a proximal end of the medical device and an end effector positioned at a distal end of the medical device. The end effector may be receivable within the working channel. The end effector may include a vessel. The vessel may include an opening. The end effector may include a blade. The blade may include a first sharp edge on a first side and a second sharp edge on a second side opposite the first side. The end effector may include a first configuration and a second configuration different from the first configuration. When the end effector is in the first configuration, the first sharp edge may extend from one side of the opening. When the end effector is in the second configuration, the second sharp edge may extend from another side of the opening. Actuating the actuator of the medical device may transition the end effector from the first configuration to the second configuration.

### BRIEF DESCRIPTION OF THE FIGURES

The accompanying drawings, which are incorporated in and constitute a part of this specification, illustrate various aspects of this disclosure and together with the description, serve to explain the principles of the disclosure.

FIG. 1 depicts exemplary medical system, including an exemplary medical device, along with an enlarged view of a distal portion of the medical system.

FIG. 2A depicts a side view of the medical device of FIG. 1 and further depicts the medical device approaching a treatment site.

FIG. 2B depicts a side view of the medical device of FIG. 1 and further depicts the medical device positioned on a portion of the treatment site.

FIG. 2C depicts a side view of the medical device of FIG. 1 and further depicts the medical device being removed from the treatment site.

FIG. 3A depicts a side view of an exemplary medical device and further depicts the medical device approaching a treatment site.

FIG. 3B depicts a side view of the medical device of FIG. 3A and further depicts the medical device positioned on a portion of the treatment site.

FIG. 3C depicts a side view of the medical device of FIG. 3A and further depicts the medical device being removed from the treatment site.

FIG. 4A depicts a side view of an exemplary medical device and further depicts the medical device approaching a treatment site.

FIG. 4B depicts a side view of the medical device of FIG. 4A and further depicts the medical device positioned on a portion of the treatment site.

FIG. 4C depicts a side view of the medical device of FIG. 4A and further depicts the medical device being removed from the treatment site.

### DETAILED DESCRIPTION OF THE FIGURES

Particular aspects of the disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of ±10% in a stated numeric value or range.

This disclosure is drawn to medical devices such as tissue removal devices, and related methods of use thereof.

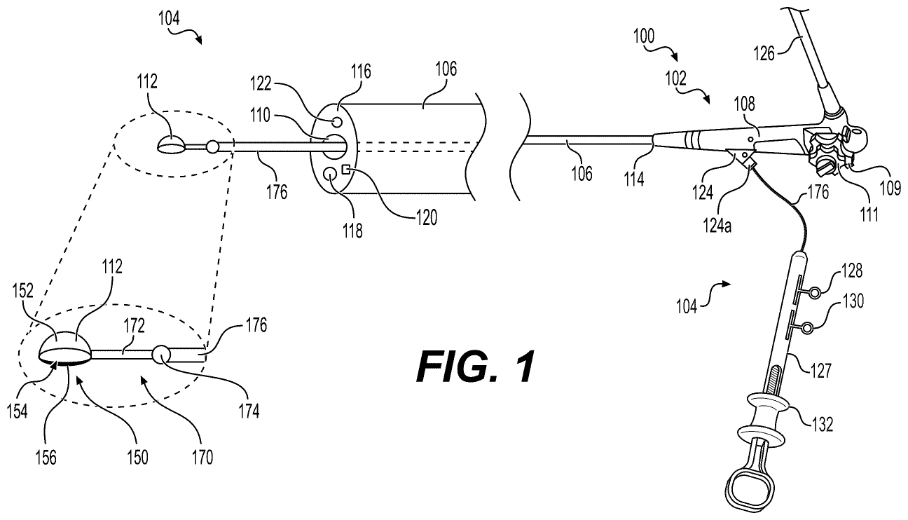

FIG. 1 depicts various aspects of an exemplary medical system 100, including an insertion device 102 and a medical device 104. Insertion device 102 includes a shaft 106, and insertion device 102 may include a handle 108. A proximal end of shaft 106 may extend from a distal end of handle 108. Shaft 106 includes one or more working channels 110, which may be fluidly connected to a port 124 on handle 108. Medical device 104 may be delivered to a treatment site via insertion device 102, for example, through port 124 and working channel 110. Additionally, medical device 104 includes an end effector 112 at a distal end of medical device 104 that may be manipulated to transition between at least a first configuration and a second configuration. The first configuration and second configuration will be explained in more detail below. Furthermore, medical device 104 may include one or more conductive portions, for example, to help mark tissue. In these aspects, and as discussed in detail below, medical device 104 may be extended (e.g., from insertion device 102) and manipulated to mark a treatment site (e.g. the tissue surrounding a polyp) surrounding a target (e.g., a polyp or other tissue). Medical device 104 may include a handle 127 positioned at a proximal end of medical device 104.

Handle 108 may be positioned at a proximal end 114 of shaft 106. Shaft 106 may be generally tubular, and may extend from a distal end of handle 108. Shaft 106 includes a distal end/distal face 116. One or more working channels 110 may extend at least from proximal end 114 of shaft 106 (e.g., that is adjacent to the distal end of handle 108) to distal end 116 of shaft 106. Shaft 106 may define multiple working channels, one or more fluid channels, and/or one or more lumens. Working channel 110 may be sized and shaped to receive at least a portion of end effector 112. Working channel 110 may include a distal opening at distal end 116. Distal end 116 of shaft 106 may include an illumination device or light 118 and/or may include a visualization device or camera 120. Distal end 116 may include one or more fluid channels 122. Fluid channel(s) 122 may be a fluid delivery channel and/or may be a suction or negative pressure channel. Handle 108 may include other types of valves, such as, for example, fluid supply valves (e.g., air and/or water valves), and/or valves that perform a combination of function such as, for example, fluid supply valves (e.g., air and/or water valves), and/or valves that perform a combination of functions. An operator may connect handle 108 to a source of suction, and may operate the suction valve to control suction through insertion device 102 (e.g., through working channel 110 or other lumen).

As mentioned, handle 108 may include port 124. Port 124 and an opening 124a of port 124 may be in communication with working channel 110. An operator may insert one or more instruments (such as end effector 112) into port opening 124a of port 124 and may extend the instrument(s) distally through working channel 110. The operator may exchange end effector 112 with a different instrument by removing end effector 112 from working channel 110 and port 124, and then inserting and extending the different instrument through port 124 and working channel 110.

Still referring to FIG. 1, an umbilicus 126 may extend from handle 108 and include or carry wires, cables, and/or conduits configured to provide, for example, power, signals, or fluids to and/or from handle 108. For example, umbilicus 126 may connect handle 108 to one or more user interfaces, monitors, controls units, displays, etc. As mentioned above, distal end 116 of shaft 106 may include illumination device or light 118 and/or may include visualization device or camera 120. Umbilicus 126 may be connected to illumination device or light 118 and/or visualization device or camera 120. Illumination device or light 118 may include LEDs, optical fibers (e.g., plastic optical fibers (POFs)), or other suitable light sources. Visualization device or camera 120 may be configured to take video and/or still images. Visualization device or camera 120 may provide a signal to a monitor or distal (e.g. connected via umbilicus 126), so that an operator may view an image provided by visualization device or camera 120 while navigating or positioning medical device 104, for example, through a body lumen, through an orifice, and/or relative to a treatment site. Distal end 116 may further include the one or more fluid channels 122. Umbilicus 126 may be fluidly connected to one or more fluid channels 122.

Shaft 106 may be a flexible shaft (e.g., a catheter). Handle 108 as shown may include first and second actuators 109, 111, which control articulation of shaft 106, e.g., an articulation joint at or proximate a distal end of shaft 106. For example, the actuators 109, 111 may control movement of the shaft 106 in multiple directions, e.g., movement along different planes. Actuators 109, 111, may include, for example, rotatable knobs that rotate to push/pull cables or wires coupled to shaft 106. For example, one or more cables or wires may comprise medical grade plastic or metal, and may extend distally from a respective actuator 109, 111 to connect to shaft 106 to control movement thereof. Distal ends of the cables or wires may extend through shaft 106 and terminate at an articulation joint and/or a distal tip of shaft 106. For example, one or more cables or wires may be connected to an articulation joint, and rotation of actuators 109, 111 may control the cables or wires to move the articulation joint and/or the distal end of shaft 106, e.g., along multiple directions. According to some aspects of this disclosure, one or more electrical cables (not shown) may extend from the proximal end of system 100 to the distal end of shaft 106 and may provide electrical controls to imaging, lighting, and/or other electronics at the distal end of shaft 106. Electrical cables may carry imaging signals received at the distal end of shaft 106 to be processed and/or displayed on a display.

Still referring to FIG. 1, handle 127 of medical device 104 may include one or more actuators, for example, three actuators 128, 130, 132. Although three actuators 128, 130, 132 are depicted, it will be appreciated that alternative numbers of actuators (e.g. one, two, four or more actuators) may be utilized. One or more actuators 128, 130, 132 may be knobs. One or more actuators 128, 130, 132 are not limited to knobs and may be levers, buttons, sliders, or any other suitable actuator. Each actuator 128, 130, 132 may control (e.g., be coupled to) a cable or wire connected to the respective actuator 128, 130, 132. The cable or wire connected to each actuator 128, 130, 132 may be directly or indirectly connected to a portion of end effector 112 and/or medical device 104. Actuating one or more of actuators 128, 130, 132 may move or otherwise manipulate one or more cables proximally or distally and thereby actuate a component/portion of end effector 112 as will be explained in greater detail in later sections.

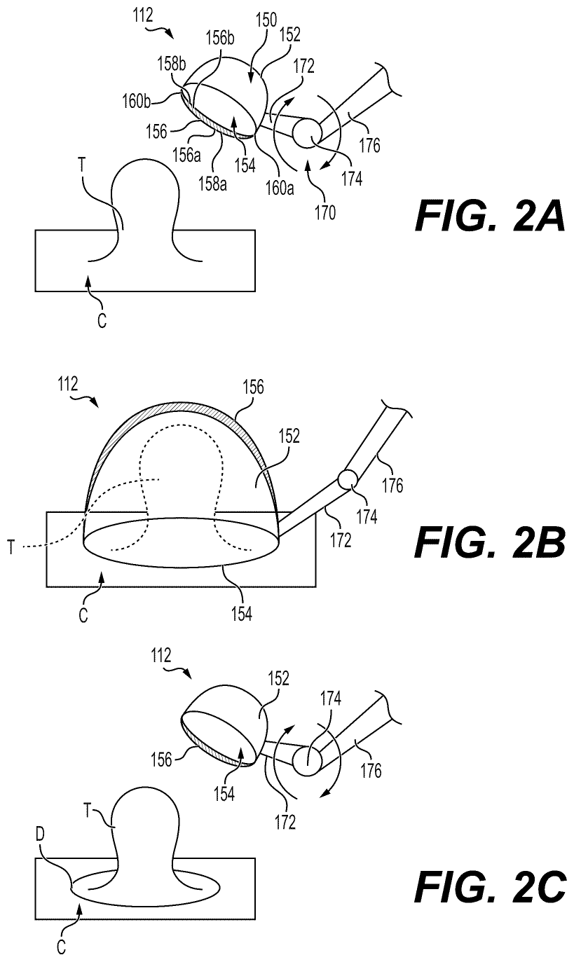

As shown in FIGS. 1 and 2A-2C medical device 104 includes end effector 112. End effector 112 may include a receiving portion 150 and a control or articulation portion 170. Receiving portion 150 may include a vessel 152. Vessel 152 may include a semi-spherical shape, however, vessel 152 is not limited to including a semi-spherical shape and may include any shape such as a semi-ellipsoid, semi-cylinder, or a prism. Vessel 152 may include an opening 154. Opening 154 may include a circular shape, however, opening 154 is not limited to a circular shape and may include any shape such as an ellipse, rectangle, or other shape. Vessel 152 may be sized and shaped to receive a target T and a portion of treatment site C (FIGS. 2A-2C). In some aspects, target T may be a polyp, and treatment site C may be a portion of a colon.

Receiving portion 150 may include a blade 156. Blade 156 may include an arcuate shape. Blade 156 may be sized and shaped to extend along a portion of an inner perimeter of opening 154. For example, blade 156 may be sized and/or shaped to extend along approximately one half of an inner surface of opening 154. Blade 156 may include a first side 156a and a second side 156b. Sides 156a, 156b may be positioned opposite one another. First side 156a may include a first sharp edge 158a. First sharp edge 158a may extend along a portion or an entirety of a length of first side 156a. Second side 156b may include a second sharp edge 158b. Second sharp edge 158b may extend along a portion or an entirety of a length of second side 156b. First sharp edge 158a and second sharp edge 158b may each be configured to at least partially cut or mark tissue.

Blade 156 may include a first longitudinal end 160a and a second longitudinal end 160b. First longitudinal end 160a may be positioned opposite of second longitudinal end 160b. First longitudinal end 160a may be positioned near or at and pivotally connected to a proximalmost portion of opening 154. Second longitudinal end 160b may be positioned near or at a distalmost portion of opening 154. In some examples, vessel 152 may include one or more hinges (not shown) to facilitate first longitudinal end 160a and second longitudinal end 160b pivoting relative to opening 154.

As illustrated in FIGS. 2A-2C, blade 156 may be operably connected to and actuated via one of actuators 128, 130, 132. Blade 156, when actuated, may be pivotable to transition between at least a first configuration and a second configuration. In the first configuration, blade 156 is positioned at one side or end of opening 154, and, in the second configuration, blade 156 is positioned at another (e.g., opposite) side or end of opening 154. In the first configuration, first sharp edge 158a may protrude from opening 154 (e.g., downward in FIGS. 2A-2C), so that first sharp edge 158a contacts treatment site C when end effector 112 is pressed against treatment site C. In the second configuration, second sharp edge 158b may protrude from opening 154 (e.g., downward in FIGS. 2A-2C), so that second sharp edge 158b contacts treatment site C when end effector 112 is pressed against treatment site C. As shown in FIGS. 2A-2C, as blade 156 transitions from the first configuration to the second configuration, blade 156 may move or travel along an interior surface of vessel 152 while pivoting about opening 154 (e.g., with hinges coupling blade 156 at a proximalmost point of opening 154 and at a distalmost point of opening 154). Handle 127 may include one or more internal springs or biasing mechanisms (not shown), for example, to bias one or more actuators 128, 130, 132, and thus, portions of end effector 112, to a neutral or original position. For example, the internal springs or biasing mechanisms may return blade 156 from the second configuration to the first configuration.

Articulation portion 170 may include an arm 172. Arm 172 may be connected to a distal portion of vessel 152 at a distal end of arm 172. Articulation portion 170 may include a pivot 174 positioned at a proximal end of arm 172. Articulation portion 170 be positioned at a distal end of a shaft 176 of end effector 112. A distal portion of shaft 176 may be connected to pivot 174. Arm 172 and shaft 176 may each include an internal lumen (not shown) sized and shaped to contain one or more wires, cables, or electrical conduits. Arm 172 may pivot about pivot 174 along one or more directional planes such as left-and-right and up-and-down in response to actuating one or more actuators 128, 130, 132. For example, actuating one of actuators 128, 130, 132 may pivot arm 172 left and right (e.g. may pivot arm on a horizontal plane) and actuating a different one of actuators 128, 130, 132 may pivot arm 172 up and down (e.g., may pivot arm 172 along a vertical plane). Additional actuators, in addition to actuators 128, 130, 132, may be included and may actuated to rotate arm 172 at pivot 174, such as, for example, along a longitudinal axis of arm 172 or a longitudinal axis of medical device 104.

FIGS. 2A, 2B, and 2C show medical device 104 being used in an exemplary procedure. During the procedure, the operator may navigate insertion device 102 (FIG. 1) of medical system 100 near target T and treatment site C. After the operator has navigated insertion device 102 near target T, the operator may extend end effector 112 from working channel 110 until end effector 112 is near target T, as shown in FIG. 2A. The operator may articulate end effector 112 left and/or right via an actuator (for example, actuator 128) and up and down via another actuator (for example, actuator 130) in order to position end effector 112 as shown in FIG. 2B. FIG. 2B shows end effector 112 positioned so that target T is received by opening 154 and vessel 152. Additionally, FIG. 2B illustrates opening 154 and/or blade 156 contacting a portion of treatment site C. The operator may actuate end effector 112 via actuators 128, 130 or by actuating/moving insertion device 102, so that the first sharp edge 158a of blade 156 cuts into treatment site C and forms first portion of a marker D (for example, the first portion of the marker may be an arcuate shape similar or identical to the shape of blade 156). Marker D may be a cut or a wound formed in treatment site C.

After forming the first portion of marker D with first sharp edge 158a, the operator may actuate blade 156, for example, via actuator 132, so that blade 156 transitions from the first configuration to the second configuration. In the second configuration, second sharp edge 158b of blade 156 cuts into treatment site C and forms a second portion of a marker D. The first and second portions of marker D may form marker D, marker D may include a circular or elliptical shape. Marker D is not limited to including a circular or elliptical shape. After marker D has been formed in treatment site C, the operator may deactuate actuator 132 so that blade 156 transitions back from the second configuration to the first configuration, as shown in FIG. 2C. It should be understood that the operator may reposition end effector 112 as needed and may form additional markers D, and portions thereof, as needed. Additionally, after marker D has been formed in treatment site C, the operator may articulate end effector 112 with the actuator and the other actuator in order to remove end effector 112 from target T and treatment site C. After the operator has removed end effector 112 from target T and treatment site C, end effector 112 may be retracted into working channel 110 and then removed through port 124. After end effector 112 has been removed from working channel 110 and port 124, a different instrument (such as a snare device, hemostatic forceps, scissors, graspers, baskets, probes, retrieval devices, lasers, and other tools) may be inserted and extended through port 124 and working channel 110, for example, to remove or otherwise treat target T (e.g., without requiring the removal of insertion device 102 from the bodily lumen). The operator may then remove target T with the different medical instrument and utilize marker D to help determine if the entirety of target T has been removed. If a portion of marker D remains after target T has been removed, the remaining portion of marker D may alert the operator that a portion of target T has not been removed.

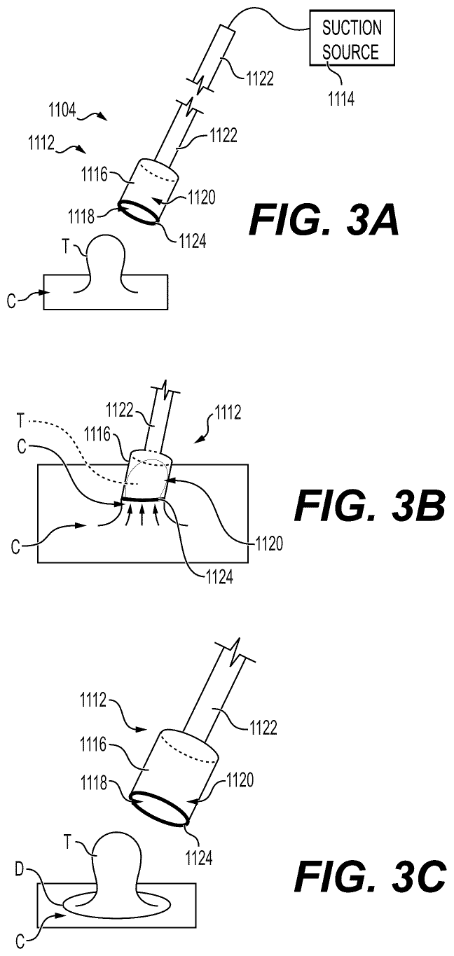

FIGS. 3A-3C depict various aspects of an exemplary medical device 1104. A distal portion of medical device 1104 may be delivered to treatment site C with an insertion device (not shown, such as insertion device 102). Medical device 1104 may include an end effector 1112 at a distal end of medical device 1104. Medical device 1104 may include or otherwise be configured to be coupled to a suction source 1114. Suction source 1114 may be configured to apply suction or negative pressure to end effector 1112. Suction source 1114 may be coupled to a handle (not shown) and/or a shaft 1122 of medical device 1104.

End effector 1112 may include a container 1116. Container 1116 may include an opening 1118, for example, positioned at a distal end of container 1116. Container 1116 may include a cylindrical shape. However, it is noted that container 1116 is not limited to a cylinder shape, and may be any shape. Furthermore, opening 1118 may be a side opening, for example, positioned on a side of container 1116 proximal of the distal end. Container 1116 may include a chamber 1120. In some aspects, chamber 1120 may be sized and/or shaped to receive at least a portion of target T and/or a portion of a treatment site C. As mentioned, medical device 1104 includes shaft 1122, and a distal end of shaft 1122 may be connected to container 1116 (e.g., at a proximal end of container 1116) or to end effector 1112. Shaft 1122 may include an internal lumen (not shown). The internal lumen of shaft 1122 may be in communication with chamber 1120 and suction source 1114. The internal lumen of shaft 1122 may be configured to apply suction or negative pressure to chamber 1120 via suction source 1114. End effector 1112 may be formed from plastic, metal, or other suitable material.

Opening 1118 may include a rim 1124 extending along a perimeter or outer edge of opening 1118. Prior to using end effector 1112 in a medical procedure, rim 1124 may be coated with a dye, marker pigment, or one or more other materials that may be applied to treatment site C. The dye or marker pigment may be configured to transfer to tissue (e.g., portions of treatment site C) upon contact of rim 1124 with the tissue. The dye or marker pigment may include indigo carmin, methylene blue, and/or other dyes or marker pigments utilized by those skilled in the art.

FIGS. 3A, 3B, and 3C show medical device 1104, including end effector 1112, being used in an exemplary procedure. Prior to using end effector 1112 in a procedure, opening 1118 and/or rim 1124 may be coated in the dye or marker pigment. For example, an operator may coat opening 1118 and/or rim 1124 prior to inserting end effector 1112 through a port and working channel of an insertion device. Alternatively, opening 1118 and/or rim 1124 may be pre-coated prior to use. In any of these aspects, the operator may navigate the insertion device (e.g., insertion device 102) to a position near target T and treatment site C. End effector 1112 may then be extended distally from a working channel of the insertion device, so that end effector 1112 is positioned near target T and treatment site C, as shown in FIG. 3A. The operator may then position end effector 1112 so that at least a portion of target T is adjacent to opening 1118, received by opening 1118, and/or contained within chamber 1120.

As shown in FIG. 3B, the operator may then utilize suction source 1114 to create or otherwise apply negative pressure within chamber 1120. The negative pressure within chamber 1120 may draw more of target T within chamber 1120 and/or cause rim 1124 to contact treatment site C. As rim 1124 contacts treatment site C, the dye or marker pigment coating rim 1124 may be deposited onto/injected into treatment site C to form a marker D on treatment site C. Marker D may be the same shape as rim 1124 and may surround target T, as shown in FIG. 3C. After marker D has been applied the treatment site, the operator may cease applying suction to chamber 1120 via suction source 1114. The operator may also manipulate or reposition end effector 1112, for example, such that target T is no longer within chamber 1120.

After the operator has removed end effector 1112 from target T and treatment site C, end effector 1112 may be retracted into a working channel (e.g., working channel 110) of an insertion device (e.g., insertion device 102) and then removed through a port of a handle of the insertion device. After end effector 1112 has been removed from the working channel and port, a different instrument (such as a snare device, hemostatic forceps, scissors, graspers, baskets, probes, retrieval devices, lasers, and other tools) may be inserted and extended through the port and working channel, for example, to remove or otherwise treat target T (e.g., without requiring the removal of insertion device 102 from the bodily lumen). The operator may then remove target T with the different medical instrument and utilize marker D to help determine if the entirety of target T has been removed. If a portion of marker D remains after target T has been removed, the remaining portion of marker D may alert the operator that a portion of target T has not been removed.

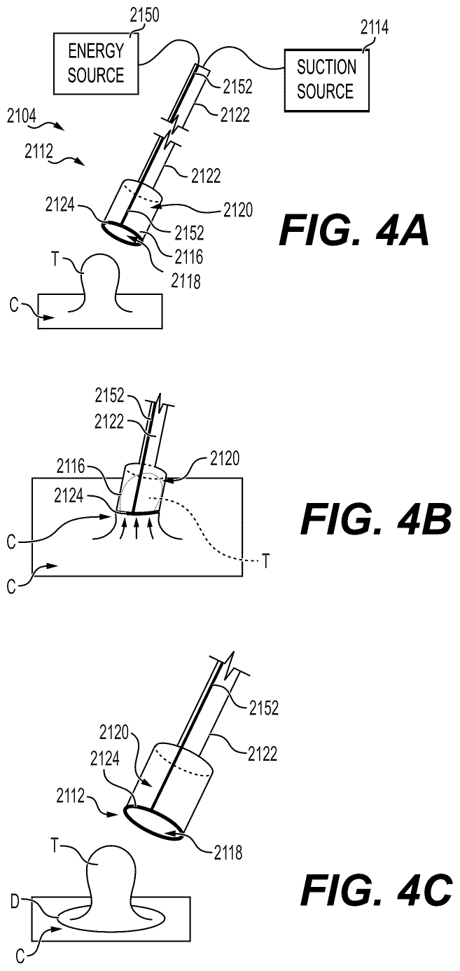

FIGS. 4A-4C depict aspects of an exemplary medical device 2104. Medical device 2104 may include any of the features of medical device 1104, except as noted below. For example, medical device 2104 may be delivered through an insertion device (e.g., insertion device 102). Medical device 2104 may include an end effector 2112. Medical device 2104 may be configured to be coupled to a suction source 2114. Suction source 2114 may be configured to apply suction or negative pressure to end effector 2112. Medical device 2104 may be configured to be coupled to an energy source 2150. Energy source 2150 may be configured to deliver electrical current/power to end effector 2112, for example, via an electrical conduit 2152. Electrical conduit 2152 may be one or more wires or cables.

As shown in FIGS. 4A-4C, a container 2116 of end effector 2112 may include an opening 2118. Opening 2118 may include a rim 2124, and at least a portion of rim 2124 may be conductive. Rim 2124 may be energized by energy source 2150 via electrical conduit 2152. Rim 2124 may be energized with RF energy or otherwise heated. Electrical conduit 2152 may extend from energy source 2150 along or within a shaft 2122 of medical device 2104 to rim 2124. Electrical conduit 2152 may extend along an exterior surface of shaft 2122, or may extend within an interior lumen of shaft 2122. Additionally, although not shown, one or more portions of electrical conduit 2152 may include one or more insulation layers.

FIGS. 4A, 4B, and 4C show medical device 2104 being used in an exemplary procedure. During the procedure, the operator may navigate the insertion device (e.g., insertion device 102) to a position near target T on treatment site C. End effector 2112 may then be extended distally from a working channel of the insertion device, so that end effector 2112 is positioned near target T and treatment site C. The operator may then position end effector 2112 so that at least a portion of target T is adjacent to opening 2118 (e.g., as shown in FIG. 4A), received by opening 2118, and/or contained within chamber 2120 of container 2116. As shown in FIG. 4B, the operator may then utilize suction source 2114 to create or apply negative pressure within chamber 2120. The negative pressure within chamber 2120 may draw more of target T within chamber 2120 and/or cause rim 2124 to contact treatment site C.

The operator may energize rim 2124 via energy source 2150 and electrical conduit 2152. The operator may press or otherwise apply energized rim 2124 into treatment site C. While in contact with treatment site C, energized rim 2124 may burn, ablate, cauterize, or otherwise mark the tissue of treatment site C surrounding target T, forming marker D. Marker D may be the same shape as rim 2124, and may surround target T, as shown in FIG. 4C. After marker D has been applied to the treatment site, the operator may de-energize rim 2124 and/or may cease applying suction to chamber 2120 via suction source 2114 and remove target T from chamber 2120.

After the operator has removed end effector 2112 from target T and treatment site C, end effector 2112 may be retracted into a working channel (e.g, working channel 110) of an insertion device (e.g., insertion device 102) and then removed through a port of a handle of the insertion device. After end effector 2112 has been removed from the working channel and port, a different instrument (such as a snare device, hemostatic forceps, scissors, graspers, baskets, probes, retrieval devices, lasers, and other tools) may be inserted and extended through the port and working channel, for example, to remove or otherwise treat target T (e.g., without requiring the removal of insertion device 102 from the bodily lumen). The operator may then remove target T with a different medical instrument (such as forceps) and utilize marker D to help determine if the entirety of target T has been removed. If a portion of marker D remains after target T has been removed, the remaining portion of marker D may alert the operator that a portion of target T has not been removed.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

What is claimed is:

1. A medical device comprising:
a receiving portion, the receiving portion comprising:
a vessel, the vessel including an opening,
a blade, the blade including a first sharp edge on a first side and a second sharp edge on a second side opposite to the first side;
wherein the receiving portion includes a first configuration and a second configuration;
wherein, when the receiving portion is in the first configuration, the first sharp edge extends from one side of the opening;
wherein, when the receiving portion is in the second configuration, the second sharp edge extends from another side of the opening.

2. The medical device of claim 1, wherein the blade is pivotable about a proximalmost point of the opening and a distalmost point of the opening.

3. The medical device of claim 2, wherein the blade is operably connected to an actuator, wherein actuating the actuator transitions the receiving portion from the first configuration to the second configuration.

4. The medical device of claim 3, wherein the blade travels along an interior surface of the vessel while the receiving portion transitions from the first configuration to the second configuration.

5. The medical device of claim 1, wherein, when in the first configuration, the blade is configured to cut a portion of a marker into a treatment site.

6. The medical device of claim 5, wherein, when in the second configuration, the blade is configured to cut another portion of the marker into the treatment site.

7. The medical device of claim 6, wherein the marker includes a circular shape or an elliptical shape.

8. The medical device of claim 1, wherein the medical device includes an articulation portion comprising:
a pivot; and
an arm;
wherein a proximal end of the arm is pivotally connected to the pivot, and wherein a distal end of the arm is connected to a proximal end of the vessel.

9. The medical device of claim 8, wherein the pivot is operably connected to a first actuator, wherein actuating the first actuator pivots the arm along a horizontal plane.

10. The medical device of claim 9, wherein the pivot is operably connected to a second actuator, wherein actuating the second actuator pivots the arm along a vertical plane.

11. The medical device of claim 10, wherein the receiving portion is operably connected to a third actuator, wherein actuating the third actuator transitions the receiving portion from the first configuration to the second configuration, wherein deactuating the third actuator transitions the receiving portion from the second configuration to the first configuration.

12. The medical device of claim 1, wherein the vessel includes a semi-spherical shape or semi-ellipsoidal shape.

13. The medical device of claim 12, wherein the opening includes a circular or elliptical shape.

14. The medical device of claim 12, wherein the blade includes an arcuate shape.

15. The medical device of claim 14, wherein a first longitudinal end of the blade is pivotally connected to a proximalmost point of the opening and a second longitudinal end of the blade is pivotally connected to a distalmost point of the opening.

16. A medical device comprising:
a container, the container including a chamber and an opening at a distal end of the chamber, the opening including a rim extending along a perimeter of the opening; and
a shaft, wherein a distal end of the shaft is connected to a proximal end of the container;
wherein the rim is configured to apply a material or deliver energy to form a marker on a treatment site surrounding a target.

17. The medical device of claim 16, wherein the medical device is configured to be coupled to a suction source, wherein the shaft includes an internal lumen that fluidly connects the suction source to the chamber of the container, and wherein the suction source is configured to create negative pressure within the chamber of the container.

18. The medical device of claim 17, wherein the rim is coated in a dye or marker pigment, and wherein the marker is formed by the dye or marker pigment.

19. The medical device of claim 16, wherein the medical device includes an electrical conduit that is electrically connected to the rim, wherein the electrical conduit is configured to be coupled to an energy source, and wherein the rim is configured to be energized by energy delivered by the electrical conduit.

20. A medical system comprising:
an insertion device, wherein the insertion device includes:
a handle, the handle including a port; and
a shaft, the shaft extending from a proximal end of the handle, the shaft including a working channel; and
a medical device, wherein the medical device includes an actuator positioned at a proximal end of the medical device and an end effector positioned at a distal end of the medical device, the end effector being receivable within the working channel, the end effector comprising:
a vessel, the vessel including an opening, and
a blade, the blade including a first sharp edge on a first side and a second sharp edge on a second side opposite to the first side;
wherein the end effector includes a first configuration and a second configuration different from the first configuration;
wherein, when the end effector is in the first configuration, the first sharp edge extends from one side of the opening;
wherein, when the end effector is in the second configuration, the second sharp edge extends from another side of the opening;
wherein actuating the actuator of the medical device transitions the end effector from the first configuration to the second configuration.
